(19) United States
(12) Patent Application Publication
(10) Pub. No.: US 2023/0356253 A1
(43) Pub. Date: Nov. 9, 2023

(54) SMALL ACTIVE AREA PLATE EJECTOR FOR DROPLET DELIVERY DEVICE

(71) Applicant: Pneuma Respiratory, Inc., Boone, NC (US)

(72) Inventors: Michael Scoggin, Boone, NC (US); Jeffrey Miller, Boone, NC (US); Caley Modlin, Boone, NC (US); Chao-Ping Lee, Kaohsiung (TW); Jianqiang Li, Shenzhen (CN)

(73) Assignee: Pneuma Respiratory, Inc., Boone, NC (US)

(21) Appl. No.: 18/311,710

(22) Filed: May 3, 2023

Related U.S. Application Data
(60) Provisional application No. 63/337,885, filed on May 3, 2022.

(51) Int. Cl. B05B 17/00; B05B 1/08

(52) U.S. Cl. CPC B05B 17/0646; B05B 1/083

(57) ABSTRACT

A droplet delivery device includes an ejector plate having an overall area that includes an outer area that is solid without holes and inner, active area with holes.

### CROSS-REFERENCE TO RELATED APPLICATIONS

This application claims the benefit of priority of U.S. Provisional Patent Application No. 63/337,885 filed May 3, 2022, which is incorporated herein by reference in its entirety.

### FIELD OF THE INVENTION

This disclosure relates to improved plate ejectors for droplet delivery devices.

### BACKGROUND OF THE INVENTION

The use of droplet generating devices for the delivery of substances to the respiratory system is an area of large interest. A major challenge is providing a device that delivers an accurate, consistent, and verifiable amount of substance, with a droplet size that is suitable for successful delivery of substance to the targeted area of the respiratory system.

Droplet delivery devices include an ejector mechanism with a mesh or plate that creates droplets from liquid passing through holes in the mesh when a powered transducer acts on the liquid and ejector mechanism. In some devices a membrane may be oscillated by a powered transducer to push the liquid through the mesh and create droplets (“push mode”), while in other devices a transducer can be coupled directly to oscillate the mesh to create droplets.

Droplet delivery devices can be used for promoting inhalation of numerous therapeutic substances (e.g. pharmaceutical and medicinal) and non-therapeutic substances (e.g. nicotine and cannabinoids). Improving and optimizing aerosolization by giving greater control over the Median Mass Aerodynamic Diameter (MMAD) of the desired droplets continues to be an area of need.

### SUMMARY OF THE INVENTION

In embodiments, the present invention includes an ejector plate for a droplet delivery device that has a smaller active “area” within the overall area of an ejector plate.

In further embodiments, the smaller active area of the plate ejector including holes with a further “solid,” i.e., no holes, outer surrounding plate area and avoids liquid from flowing through the holes of a conventional plate. i.e., without a solid outer area, passing through an ejector plate and residing on the surface of the plate. Instead, the smaller active area promotes efficient aerosolization through more precise control of MMAD.

In certain embodiments an ejector plate includes a raised feature, such as including a dome shape, in combination with an active area of including holes. In embodiments, the raised featured is centrally positioned in or on the ejector plate.

In embodiments of the invention, an anulus is coupled to an ejector plate that includes an active area with holes and a solid area without holes.

### BRIEF DESCRIPTION OF THE DRAWINGS

FIG. 1 illustrates a top plan view of a typical ejector plate having holes throughout the full area of the ejector plate (prior art).

FIG. 2 illustrates a top plan view of an ejector plate having holes in a small partial plate area of the ejector plate in an embodiment of the invention.

FIG. 3A is a side plan view of an ejector plater including a dome or raised center area in an embodiment of the invention.

FIG. 3B is a top plan view of an ejector plater including a dome or raised center area with an outer “active area” bordering the dome or raised center area in an embodiment of the invention

### DETAILED DESCRIPTION

The present application incorporates the contents of (a) U.S. patent application Ser. No. 17/846,902 filed Jun. 22, 2022, entitled “Droplet Delivery Device with Push Ejection,” (b) International Publication Number WO 2020/264501 published Dec. 30, 2020, entitled “DELIVERY OF SMALL DROPLETS TO THE RESPIRATORY SYSTEM VIA ELECTRONIC BREATH ACTUATED DROPLET DELIVERY DEVICE” and (c) International Publication Number WO 2020/227717 published Nov. 12, 2020, entitled “ULTRASONIC BREATH ACTUATED RESPIRATORY DROPLET DELIVERY DEVICE AND METHODS OF USE, together herein by reference in their entirety (including such publications and patent applications, herein also included by reference in their entirety, as are cited and incorporated by reference or relied upon in the foregoing disclosures). Ejector plates with smaller “active areas” having holes with a bordering area of an ejector plate being solid with no holes may be used in devices such as those disclosed in the foregoing incorporated references.

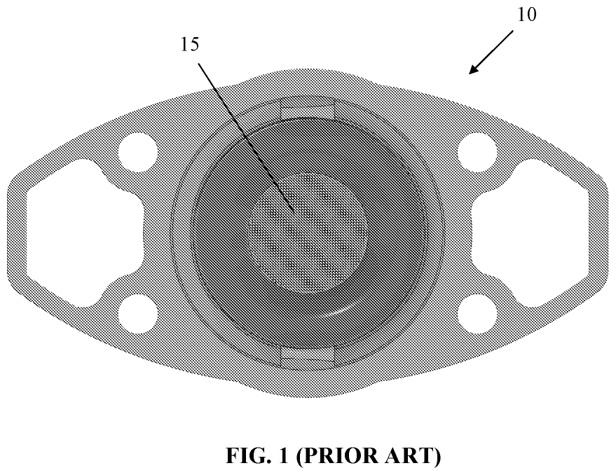

Referring to FIG. 1, a typical ejector 10 includes an ejector plate 15 entirely full of holes. The holes on the outer area of the ejector plate 15 do not assist in the aerosol generation. During vibration, such as by an electrically powered transducer coupled to the ejector plate, including a piezoelectric transducer in some embodiments, there is not enough energy for the outer area hole to produce aerosol. Instead, liquid flows through the holes and resides on the surface of the ejector plate 15. This liquid is aerosolized through the vibration of the ejector plate 15 resulting in larger droplets, greater than 10 micron in diameter. The aerosol generated through the normal aerosolization through the holes is in a defined range i.e., 1-2 micron, or 2-3 micron, etc.

For the ejector plate shown in FIG. 1, the outer area holes do not create aerosol, instead liquid comes through the hole and rests on top of the ejector plate. Then, the liquid is aerosolized into large droplets, greater than 10 microns.

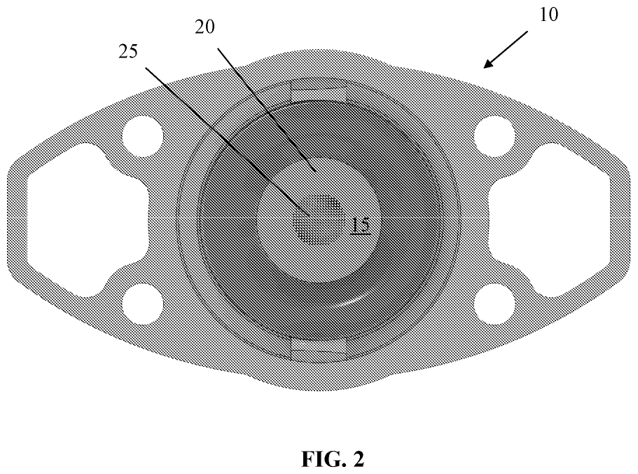

Referring to FIG. 2, to mitigate the creation of larger droplets by ejector 10, a smaller “active area” 25 of holes is made on the ejector plate 15. This is done by leaving the outer area 20 of the ejector plate 15 solid, i.e., without holes, In embodiments, the active area 25 can preferably be 1 mm, 1.5 mm, 2 mm, 2.5 mm, 3 mm, or 3.5 mm in diameter, FIG. 2. This small active area 25 including holes surrounded by a solid outer ring portion 20 of the ejector plate 15 promotes only the holes of small active area 25 as generating aerosol since the outer area 20 is solid and has no holes so that no liquid flows through the solid outer area 20 to otherwise reside on the surface of the ejector plate 15 if such are 20 included holes as in conventional aperture plates of aerosol ejectors. In embodiments, the ejector plate 15 with small active area 25 having holes is coupled to an electrically powered transducer, including a piezoelectric transducer in some embodiments, to cause the ejector plate to vibrate. This configuration causes the geometric standard deviation (GSD) of the aerosol to be smaller by decreasing the range of droplet sizes generated. Having a smaller active area ejector plate also helps provide precise control over the MMAD of the aerosol.

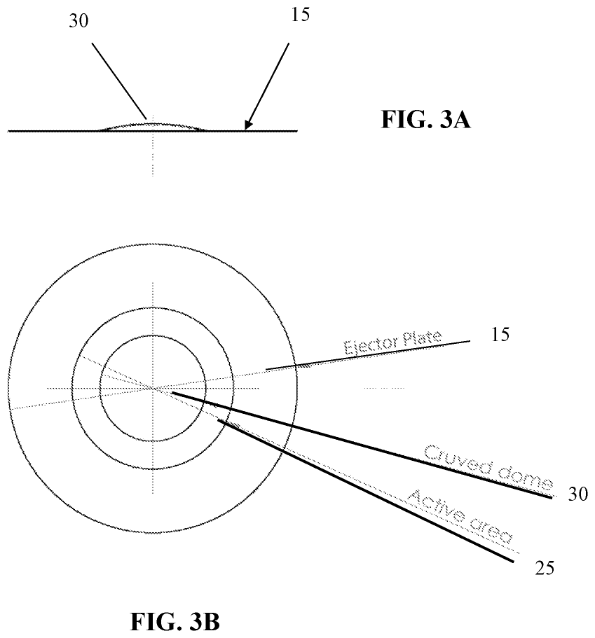

Referring to FIGS. 3A and 3B, in an embodiment, the use of a dome, or raised feature 30, is incorporated into the ejector plate 15. The dome or raised feature 30 is preferably centered on the ejector plate 15. The dome 30 forces vibrations to focus in the center of the ejector plate. The “active area” 25 including holes in the ejector plate could have an area that is greater than or less than the area of the base of the raised are or dome incorporated in the ejector plate.

In various embodiment, an anulus (not shown) is used to force the vibrations in the center of the ejector plate. The outer diameter of an anulus can be the same as the outer diameter of the ejector plate 15 to minimize the vibrations where the anulus is in contact with the ejector plate 15. The inner diameter of the anulus is preferably greater than the diameter of the “active area” 25. The anulus may or may not be connected to the ejector plate 15 via an adhesive. The use of an anulus to attenuate vibrations on the outer part of the ejector plate allows for the use of a silicone gasket to be in contact with the ejector plate and or anulus.

In embodiments the anulus is made of stainless steel or another metal. In other embodiments the anulus could be made of any material that will attenuate the vibrations in the outer edge of the ejector plate.

In another embodiment a frame-like structure is used as an alternative to an anulus. The frame-like could be made of metal in some embodiments. In other embodiments, the frame-like structure can be made of more flexible materials.

In another embodiment, duty cycle programming can be used to actuate the device and ejector plate having a smaller “active area” to vibrate in cycles. This causes vibrations to be incurred on the ejector plate in short bursts. The short bursts help eliminate chaotic vibrations that may occur from long lasting vibrations.

In other embodiments, different polymers or materials may be used in the ejector plate. A specific polymer may allow ejector vibrations to be localized only to the “active area.” The polymer could be one thickness or have different thicknesses to aid in the localization of the vibrations to an “active area” of the ejector plate.

While the invention has been described with reference to exemplary embodiments, it will be understood by those skilled m the art that various changes may be made, and equivalents may be substituted for elements thereof without departing from the scope of the invention. In addition, many modifications may be made to adapt a particular situation or material to the teachings without departing from the essential scope thereof. Therefore, it is intended that the invention not be limited to the particular embodiments, including specific sizes and shapes of ejector platers, disclosed as contemplated for carrying out this invention, but that the invention will include all embodiments falling within the scope of the appended claims.

What is claimed:

1. A droplet delivery device including an ejector plate having a solid outer area with no holes surrounding an inner active area that includes holes, wherein the ejector plate is configured to vibrate.

2. The droplet delivery device of claim 1, wherein the ejector plate is configured to vibrate via a powered transducer coupled to the ejector plate.

3. The droplet delivery device of claim 2, wherein the powered transducer is a piezoelectric transducer.

4. The droplet delivery device of claim 3, wherein the ejector plate includes a raised feature.

5. The droplet delivery device of claim 4, wherein the raised feature has a dome shape.

6. The droplet delivery device of claim 1, wherein the ejector plate is coupled to an anulus.

7. The droplet delivery device of claim 2, wherein the ejector plate is coupled to an anulus.

8. The droplet delivery device of claim 3, wherein the ejector plate is coupled to an anulus.

9. The droplet delivery device of claim 4, wherein the ejector plate is coupled to an anulus.

10. The droplet delivery device of claim 5, wherein the ejector plate is coupled to an anulus.

11. The droplet delivery device of claim 1, wherein the droplet delivery device includes a raised feature.

12. The droplet delivery device of claim 11, wherein the raised feature has a dome shape.

13. The droplet delivery device of claim 1, wherein the inner active area and solid outer area are concentric.

14. The droplet delivery device of claim 3, wherein the inner active area and solid outer area are concentric.

15. The droplet delivery device of claim 4, wherein the inner active area and solid outer area are concentric.

16. The droplet delivery device of claim 6, wherein the inner active area and solid outer area are concentric.

17. The droplet delivery device of claim 8, wherein the inner active area and solid outer area are concentric.

18. The droplet delivery device of claim 11, wherein the inner active area and solid outer area are concentric.

19. The droplet delivery device of claim 12, wherein the inner active area and solid outer area are concentric.

20. A droplet delivery device comprising:
an ejector plate with a circular inner active area having a plurality of holes and an outer solid ring area without holes surrounding the inner active area; and
a powered transducer configured to vibrate the ejector plate.
